(19) United States
(12) Patent Application Publication
(10) Pub. No.: US 2026/0165973 A1
(43) Pub. Date: Jun. 18, 2026

(54) PHARMACEUTICAL COMPOSITIONS CONTAINING DORAVIRINE AND ISLATRAVIR

(71) Applicant: MERCK SHARP & DOHME LLC, Rahway, NJ (US)

(72) Inventors: Pavithra SUNDARARAJAN, South Plainfield, NJ (US); Majid MAHJOUR, Schwenksville, PA (US); Gerard R. KLINZING, Wilmington, DE (US); Sanjaykumar PATEL, Roselle Park, NJ (US); Andrew BUSS, Allentown, PA (US); Ray Thomas MCCLAIN, Lansdale, PA (US); Laura Yvonne PFUND, East Brunswick, NJ (US); Jiaying LIU, Rahway, NJ (US); Paresh B. PATEL, Langhorne, PA (US); Daniel SKOMSKI, East Brunswick, NJ (US)

(73) Assignee: MERCK SHARP & DOHME LLC, Rahway, NJ (US)

(21) Appl. No.: 19/126,319

(22) PCT Filed: Oct. 11, 2024
(86) PCT No.: PCT/US2024/050906

§ 371 Date: May 1, 2025

Related U.S. Application Data
(60) Provisional application No. 63/589,647, filed on Oct. 12, 2023.

(51) Int. Cl. A61K 9/20

(52) U.S. Cl. CPC A61K 9/2054; A61K 9/2013; A61K 9/2027; A61K 2300/00

(57) ABSTRACT

The instant invention relates to pharmaceutical compositions comprising doravirine and islatravir. These compositions are useful for the treatment of HIV infection. Processes for making said pharmaceutical compositions are provided.

### CROSS-REFERENCE TO RELATED APPLICATIONS

This application claims the benefit of priority to U.S. Provisional Application No. 63/589,647, filed Oct. 12, 2023, the disclosure of which is incorporated herein in its entirety.

### BACKGROUND OF THE INVENTION

This invention relates to pharmaceutical compositions comprising doravirine, and islatravir. These compositions are useful for the treatment of human immunodeficiency virus (HIV) infection.

Specifically, this invention relates to single tablet fixed-dose combinations of doravirine and islatravir. Doravirine is a non-nucleoside reverse transcriptase inhibitor approved by the United States Food and Drug Administration (FDA) for the treatment of HIV infection in adult patients. It is currently available both as a single stand-alone tablet and as part of a single-tablet regimen in a fixed-dose combination with tenofovir disoproxil and lamivudine (R Talwani and Z Temesgen, Drugs Today (Barc), 2020 Feb; 56(2): 113-124).

Islatravir is a nucleoside reverse transcriptase translocation inhibitor (NRTI) being developed for the treatment and prevention of HIV-1 infection (Markowitz M and Sarafianos SG, Curr Opin HIV AIDS. 2018; 13:294-299). Islatravir inhibits reverse transcriptase (RT) through multiple mechanisms of action, including inhibiting RT translocation (Michailidis, E. et al., J. Biol. Chem. 2014, 289, 24533-24548.). This is thought to contribute to its favorable resistance profile (Maeda, K et al., Antiviral Ther. 19, 179-189), making it a potentially promising new generation NRTI.

Thus, a product combining doravirine and islatravir can be more effective for the treatment of HIV infection than either drug alone. The combination product can be particularly useful from the standpoints of both compliance and convenience.

### SUMMARY OF THE INVENTION

In part, the present disclosure provides a fixed-dose combination of doravirine and islatravir.

Based on the pharmacology of the individual compounds, a doravirine and islatravir fixed dose combination formulation would require a high dose of doravirine and a low dose of islatravir in a single dosage unit. Accordingly, novel pharmaceutical compositions that incorporate high doses of doravirine and low doses of islatravir into a compact, single-unit dosage form while maintaining robust formulation and bioperformance characteristics of co-dosed doravirine and islatravir single entity formulations are provided.

In one aspect, the fixed dose combination tablet comprises (a) from about 25% to about 80% w/w of a doravirine amorphous solid dispersion comprising doravirine and a polymer, and (b) from about 0.01% to about 0.12% w/w of islatravir.

The weight ratio of doravirine to the polymer in the solid dispersion may be about 1:4.

The above tablet may further comprise (c) from about 7.0% to about 25% w/w of lactose monohydrate; (d) from about 7.0% to about 25% w/w of microcrystalline cellulose; (e) from about 4% to about 15% w/w of a disintegrant; (f) from about 0.2% to about 1.0% w/w of a glidant; and (g) from about 0.1% to about 1.5% w/w of a lubricant; such that the lactose monohydrate and microcrystalline cellulose combined comprise about 14% to about 45% of the weight of the tablet.

The weight of the tablet may be between about 600 to about 1100 mg.

More specifically, the tablet may comprise (a) about 65% w/w of a solid dispersion comprising amorphous doravirine dispersed within a matrix formed by a polymer, such that the polymer is hydroxypropyl methyl cellulose acetate succinate (HPMCAS) and the weight ratio of doravirine to HPMCAS in the solid dispersion is about 1:4; (b) about 0.0325% w/w of islatravir; (c) about 12.48% w/w of lactose monohydrate; (d) about 12.48% w/w of microcrystalline cellulose; (e) about 9.0% w/w of croscarmellose sodium as the disintegrant; (f) about 0.5% of colloidal silicon dioxide as the glidant; and (g) about 0.5% w/w of magnesium stearate as the lubricant.

For example, the tablet may comprise (a) about 100 mg of the doravirine amorphous solid dispersion, such that the weight ratio of doravirine to HPMCAS in the solid dispersion is about 1:4; (b) about 0.25 mg of islatravir (or 0.2653 mg of islatravir monohydrate); (c) about 96 mg of lactose monohydrate; (d) about 96 mg of microcrystalline cellulose: (e) about 69.2 mg of croscarmellose sodium; (f) about 3.8 mg of colloidal silica; and (g) about 3.8 mg of magnesium stearate, such that weight of the tablet is about 769 mg.

The tablet may further comprise a film coat comprising: (a) a film former selected from the group consisting of hydroxy propyl methycellulose (Hypromellose; HPMC), hydroxyproplyl cellulose (HPC), polyvinyl alcohol (PVA), and polyethylene glycol-polyvinyl alcohol (PEG-PVA) graft copolymer; (b) a second film former selected from the group consisting of lactose, polydextrose, and isomalt; and (c) a plasticizer selected from the group consisting of triacetin, polyethylene glycol (PEG), and medium chain triglyceride; and (d) an opacifier selected from the group consisting of calcium carbonate, rice starch, magnesium carbonate, zinc oxide, magnesium oxide and titanium dioxide.

Further, the tablet may have the following dimensions: length of about 15 mm to about 21 mm and the breadth of about 7.5 mm to about 10 mm.

### BRIEF DESCRIPTION OF THE DRAWINGS

FIG. 1A is a graph showing dissolution performance of clinical formulations as percent of doravirine dissolved as a function of time for Reference (upper curve), Test A (middle curve), and Test B (lower curve).

FIG. 1B is a graph showing dissolution performance of clinical formulations as percent of islatravir dissolved as a function of time for Reference (upper curve), Test A (middle curve), and Test B (lower curve).

### DETAILED DESCRIPTION OF THE INVENTION

The novel pharmaceutical compositions of the instant invention address the need for incorporation of high doses of doravirine and low doses of islatravir into a compact, single-unit dosage form without compromising the robust formulation and bioperformance characteristics of the single entities of these compounds for treating HIV infection.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures used in preparing pharmaceutical formulations are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” means within a statistically meaningful range of a value, such as a stated concentration range, time frame, molecular weight, temperature, or pH. Such a range can be within an order of magnitude, typically within 10%, and more typically within 5% or within 1% of a given value or range (rounded up to the nearest whole number if the value is not sub-dividable, such as a number of molecules or nucleotides). Sometimes, such a range can be within the experimental error typical of standard methods used for the measurement and/or determination of a given value or range. The allowable variation encompassed by the term “about” will depend upon the system under study and can be readily appreciated by one of ordinary skill in the art. Whenever a range is recited within this application, every whole number integer within the range is also contemplated as an embodiment of the disclosure.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

In this disclosure, terms such as “comprises,” “comprised,” “comprising.” “contains,” “containing” and the like mean “includes,” “included.” “including” and the like. Such terms refer to the inclusion of particular ingredients or set of ingredients without excluding any other ingredients.

Terms such as “consisting essentially of” and “consists essentially of” allow for the inclusion of additional ingredients or steps that do not detract from the novel or basic characteristics of the disclosure, i.e., they exclude additional unrecited ingredients or steps that detract from the novel or basic characteristics of the disclosure. The terms “consists of” and “consisting of” are closed ended. Accordingly, these terms refer to the inclusion of a particular ingredient or set of ingredients and the exclusion of all other ingredients.

As used herein, “fixed dose combination” refers to a combination of defined doses of two drugs or active ingredients presented in a single dosage unit (e.g. a tablet or a capsule) and administered as such.

The term “tablet” as used herein is intended to encompass compressed pharmaceutical dosage formulations of all shapes and sizes, whether uncoated or coated.

The term “bioavailability” refers to the degree to which the pharmaceutically active agent becomes available to the target tissue after the agent's introduction into the body. Enhancement of the bioavailability of a pharmaceutically active agent can provide a more efficient and effective treatment for patients because, for a given dose, more of the pharmaceutically active agent will be available at the targeted tissue sites.

### Doravirine/Islatravir Fixed Dose Combination Tablet

As a general description, an embodiment of the instant invention comprises a single tablet that incorporates a high loading of an amorphous solid dispersion containing doravirine and a low loading of islatravir.

Doravirine is a HIV reverse transcriptase (RT) inhibitor having the chemical name 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile. Production and the ability of doravirine to inhibit HIV reverse transcriptase are illustrated in WO 2011/120133 A1 and U.S. Pat. No. 8,486,975.

Doravirine is known to exist in three crystalline anhydrous forms, designated as Form I, Form II and Form III, and in an amorphous form. Doravirine can be made into an amorphous dispersion formulation by spray-drying with or without a polymer. However, in the absence of a polymer, doravirine crystallizes readily with the crystals having a high melting point of 286° C. See International Publication No. WO 2015/077273. Neat amorphous doravirine, generated by spray drying, crystallizes within 2 weeks when stored in an open container at 5° C./ambient relative humidity (RH), 30° C./65% RH, 40° C./75% RH, and 60° C./ambient RH.

Islatravir is a nucleoside reverse transcriptase translocation inhibitor having the chemical name (2S,3R,5S)-5-(6-amino-2-fluoro-9H-purin-9-yl)-2-ethynyl-2-(hydroxymethyl)tetrahydrofuran-3-ol. Production and the anti-HIV activity of islatravir are illustrated in WO 2005/090349 A1 and U.S. Pat. No. 7,339,053. Islatravir is known to exist in several crystalline free forms, namely, monohydrate, anhydrate Form I, anhydrate Form II, anhydrate Form III and anhydrate Form IV.

The pharmaceutical compositions of the invention may contain one or more additional formulation ingredients that may be selected from a wide variety of excipients known in the pharmaceutical formulation art. According to the desired properties of the compositions, any number of ingredients may be selected, alone or in combination, based upon their known uses in preparing tablet compositions. Such ingredients include, but are not limited to, diluents, binders, compression aids, disintegrants, lubricants, glidants, stabilizers (such as dessicating amorphous silica), flavors, flavor enhancers, sweeteners, preservatives, colorants and coatings.

Accordingly, in some embodiments, the pharmaceutical compositions comprises islatravir, an amorphous dispersion formulation of doravirine, a glidant, a diluent, a disintegrant, and a lubricant.

The glidant, or flow aid used may be colloidal silica, silicon dioxide, talc, or starch. In some embodiments of the invention, the glidant is colloidal silica.

The diluents used may be lactose, lactose anhydrous, lactose monohydrate, mannitol, microcrystalline cellulose, calcium phosphate, calcium phosphate dibasic, calcium carbonate or magnesium carbonate. In some embodiments, the diluents are lactose monohydrate and microcrystalline cellulose.

The disintegrant used may be croscarmellose sodium, starch, crospovidone, sodium starch glycolate or any mixtures thereof. In some embodiments, the disintegrant is croscarmellose sodium.

The lubricant used may be magnesium stearate, stearic acid or sodium stearyl fumarate. In some embodiments, the lubricants is magnesium stearate.

In some embodiments, the pharmaceutical composition may have the following combination of excipients: colloidal silica as a glidant, lactose monohydrate and microcrystalline cellulose as diluents, croscarmellose sodium as a disintegrant, magnesium stearate as a lubricant. The pharmaceutical composition may also have a film coating. Substances which may be used for coating include hydroxypropylmethylcellulose, hydroxypropylcellulose, polyvinyl alcohol, macrogol, macrogol PVA graft copolymer, medium chain triglycerides, talc, sweeteners, and colorants. In some embodiments, the film coating is an aqueous film coating, which for example, comprises hydroxypropylmethylcellulose such as Opadry® II, Opadry® II, which is available from Colorcon, Inc., Harleysville, PA, contains hydroxypropyl methyl cellulose (also known as “HPMC” or “hypromellose”), lactose monohydrate, titanium dioxide, triacetin and colorants such as iron oxide yellow, red and black. In some embodiments, the film coating does not include titanium dioxide, such as the Opadry® TF filmcoats. Opadry® II, which is available from Colorcon, Inc., Harleysville, PA, contains hydroxypropyl methyl cellulose (also known as “HPMC” or “hypromellose”), lactose monohydrate, calcium carbonate, triacetin and colorants such as iron oxide yellow, red, and black. The pharmaceutical composition may further have a polishing aid. In some embodiments, the polishing aid is camauba wax.

Turning to doravirine spray-dried composition, several polymers may be used in the preparation of this composition including hydroxypropyl methyl cellulose acetate succinate (HPMCAS, also known as “hypromellose acetate succinate”), hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone and poly vinylpyrrolidinone-poly vinylacetate copolymers.

Amorphous dispersion formulation of doravirine has been made by spray-drying doravirine with HPMCAS-L, which significantly improves the bioavailability of doravirine. However, since doravirine is a strong crystallizer, the resulting amorphous dispersion formulation of doravirine poses many unique challenges, including physical stability. Doravirine crystallizes readily in the absence of a polymer and has a high melting point of 286° C. (see, PCT International Publication WO 2015/077273). Neat amorphous doravirine generated by spray-drying crystallizes within 2 weeks when stored in an open container at 5° C./ambient relative humidity (RH), 30° C./65% RH, 40° C./75% RH, and 60° C./ambient RH. For spray-dried dispersions of doravirine and HPMCAS, crystallization was observed at 35% drug loading after 16 weeks of storage and at 40% drug loading after 8 weeks of storage at 40° C./75% RH (open). Other factors can affect physical stability, including inherent tendency of the drug to crystallize, drug loading in the dispersion, type of polymers used, hygroscopicity of the formulation and other factors. WO 2015/077273.

In addition to challenges associated with physical stability, dissolution of the amorphous dispersion of doravirine is a concern due to its potential to reduce oral bioavailability.

Moreover, the amorphous dispersion of doravirine is associated with atypical compaction properties which gives rise to issues in processing (WO 2017095761). The compactability of doravirine spray dried dispersion is directly correlated to the bulk density of the dispersion. Higher bulk density leads to lower tensile strength tablets. Tabletability of the spray dried dispersion formulations, after roller compaction also is of concern. A relatively high roller compaction force results in lower final compactability. In certain cases, tablets of formulations containing doravirine spray dried dispersions with high bulk density show failure upon compression, due to low tensile strength (WO2015/077273).

The fixed dose combination tablet described herein addresses the observed issues related to physical stability, kinetic supersaturation effect, and processing. In addition to the above considerations that are specific to doravirine, the large difference in the dose of doravirine and islatravir presents a unique challenge for providing a robust formulation with acceptable critical attributes with regard to assay and content uniformity.

The pharmaceutical compositions of the invention are monolithic tablet formulations containing a cogranulation of islatravir and amorphous dispersion of doravirine, with islatravir being present at vastly lower amounts than doravirine. Prior to the instant invention, it could not be expected that a drug loaded at as low an amount as to constitute only 0.03% of the total weight of the formulation could be combined with another drug product intermediate at significantly higher amounts, e.g., 65%, while ensuring acceptable assay and content uniformity. The inventors of the present application achieved acceptable assay and content uniformity of islatravir based on observations made during preliminary solubilization experiments indicating that islatravir may have an affinity for polymers. The inventors found also that in the monolithic tablet containing extremely low loading of islatravir and high loadings of doravirine it was desirable, in the process of making the tablet, to introduce a comilling step between steps for blending various components to allow for islatravir to be well mixed with excipients and doravirine spray dried intermediate. Inclusion of polymer and modification of the process of making the tablet led to tablets with acceptable assay and content uniformity of islatravir. Details are provided in Example 3.

The pharmaceutical compositions of the instant invention also address the need for process robustness upon scale up. Doravirine spray dried intermediate is susceptible to cracking during tablet compression. Prototype formulation studies at high spray dried intermediate loading (greater than 50% w/w of the core tablet) revealed the risk of cracking through use of a figure of merit of maximum simulated press speed without tablet integrity failure. This figure of merit served as a surrogate measure for issues during normal compression operation. Selection of particular pharmaceutical excipients, level of the spray dried intermediate loading, and design of tooling by the inventors led to a tablet formulation that was strong and that still had acceptable dissolution performance. Details are provided in Examples 1 and 2.

The pharmaceutical compositions of the invention may be film coated to provide additional protection to guard against the photostability risks of doravirine. In this regard, the inventors developed an filmcoat which utlitizes opacifiers different from titanium dioxide (e.g., calcium carbonate) while ensuring reduced basic degradation of islatravir, physical stability of doravirine, as well as sufficient opacfication to guard against photostability risks of doravirine. Alternatives to titanium dioxide were explored due to safety issues (disputed and as yet unresolved) surrounding its use. Racovita AD, 2022, Int. J. Environ. Res. PublicHealth, 19,5681. Details are provided in Example 4.

Preliminary pharmaceutical assessment of monolithic tablet formulation of islatravir and amorphous solid dispersion of doravirine indicated that the despite high solubility of islatrivir in water and dissolution media, dissolution of islatravir in the monolithic tablet was strongly correlated with dissolution of doravirine. Doravirine is poorly soluble and is classified as a class II compound based on the biopharmaceutics classification system. To ensure that the efficacy of the fixed dose combination with respect to doravirine is comparable to the efficacy of single entity formulation of doravirine, it is important that the release of doravirine from the fixed dose combination mimic the release from the single entity formulation. It was found that for the tablets of the invention, dissolution of doravirine and islatravir in quality control dissolution methods are sensitive to variation in moisture and process parameters. However, it was found that these variations did not affect pharmacokinetics of the formulation. Prior to this finding, it would have been unexpected that allowing for moisture exposure upto 45% RH and process variations would not impact the pharmacokinetics of doravirine and islatravir. Details are provided in Example 5.

The pharmaceutical compositions of the instant invention are stable at temperatures up to 30° C. and up to 65% relative humidity for at least 24 months (including in-use exposure of at least 1 month) when stored with adequate desiccants such that the water activity of the drug product does not exceed 35%.

The pharmaceutical compositions of the instant invention includes a compact single-unit dosage form with an image size no larger than 1.1 grams comprising 100 mg of doravirine (equivalent to 500 mg of doravirine spray dried intermediate) and 0.25 mg of islatravir.

A “dosage form” in accordance with the present invention is a tablet, capsule, caplet, a multiparticulate system that includes subunits (e.g., minitablets, beads, pellets, and granules) or other solids known for the administration of medicines orally. Multiparticulates generally comprise a plurality of minitablets, beads, or pellets/granules that may range in size from about 10 μm to about ≤3 mm. more typically about 100 μm to 1 mm in diameter. Such multiparticulates may be packaged, for example, in a capsule such as a gelatin capsule or a capsule formed from a polymer such as HPMCAS, HPMC or starch; dosed in a sachet; or formed into a tablet (e.g. a caplet) or pill by compression or other processes known in the art.

The pharmaceutical composition of the invention may be provided in a daily dosing regimen. In various embodiments, the daily dosing regimen includes administration of the pharmaceutical composition once daily, twice daily, once every other day, once a week, two times a week, three times a week, four times a week, or five times a week. In some embodiments, the pharmaceutical composition is administered once daily.

### HIV Combination Therapy

The pharmaceutical compositions of the invention may be administered with at least one additional therapeutic agent. The additional therapeutic agent may be an anti-HIV agent selected from the group consisting of: a combination drug for treating HIV (HIV combination drug), other drug for treating HIV, an HIV protease inhibitor, an HIV non-nucleoside or non-nucleotide inhibitor of reverse transcriptase, an HIV nucleoside or nucleotide inhibitor of reverse transcriptase, an HIV integrase inhibitor, an HIV non-catalytic site (or allosteric) integrase inhibitor, an HIV entry inhibitor, an HIV maturation inhibitor, a latency reversing agent, a compound that targets the HIV capsid, an immune-based therapy, a phosphatidylinositol 3 kinase (PI3K) inhibitor, an HIV antibody, a bispecific antibody and an “antibody-like” therapeutic protein, an HIV p17 matrix protein inhibitor, an IL-13 antagonist, a peptidyl-prolyl cis-trans isomerase A modulator, a protein disulfide isomerase inhibitor, a complement C5a receptor antagonist, a DNA methyltransferase inhibitor, an HIV vif gene modulator, a Vif dimerization antagonist, an HIV-1 viral infectivity factor inhibitor, a TAT protein inhibitor, an HIV-1 Nef modulator, an Hck tyrosine kinase modulator, a mixed lineage kinase-3 (MLK 3) inhibitor, an HIV-1 splicing inhibitor, a Rev protein inhibitor, an integrin antagonist, a nucleoprotein inhibitor, a splicing factor modulator, a COMM domain containing protein 1 modulator, an HIV ribonuclease H inhibitor, a retrocyclin modulator, a CDK-9 inhibitor, a dendritic ICAM-3 grabbing nonintegrin 1 inhibitor, an HIV GAG protein inhibitor, an HIV POL protein inhibitor, a Complement Factor H modulator, a ubiquitin ligase inhibitor, a deoxycytidine kinase inhibitor, a cyclin dependent kinase inhibitor, a proprotein convertase PC9 stimulator, an ATP dependent RNA helicase DDX3X inhibitor, a reverse transcriptase priming complex inhibitor, a G6PD and NADH-oxidase inhibitor, a pharmacokinetic enhancer, an HIV gene therapy agent, an HIV vaccine, an HIV nucleoside reverse transcriptase translocation inhibitor, an immunomodulator, an immunotherapeutic agent, an antibody-drug conjugate, a gene modifier, a gene editor (e.g., CRISPR/Cas9, zinc finger nucleases, homing nucleases, synthetic nucleases, TALENs), a cell therapy (e.g., chimeric antigen receptor T cell (e.g., YESCARTA@ (axicabtagene ciloleucel), engineered T cell receptors, and TCR-T) and a combination thereof.

### HIV Combination Drugs

Examples of HIV combination drugs include ATRIPLA@ (efavirenz, tenofovir disoproxil fumarate, and emtricitabine); COMPLERA@ (EVIPLERA@; rilpivirine, tenofovir disoproxil fumarate, and emtricitabine); STRIBILD (elvitegravir, cobicistat, tenofovir disoproxil fumarate, and emtricitabine); TRUVADA@ (tenofovir disoproxil fumarate and emtricitabine; TDF+FTC); DESCOVY@ (tenofovir alafenamide and emtricitabine); ODEFSEY@ (tenofovir alafenamide, emtricitabine, and rilpivirine); GENVOYA@ (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR@ (zidovudine and lamivudine; AZT+3TC); EPZICOM@ (LIVEXAR; abacavir sulfate and lamivudine; ABC+3TC); KALETRA@ (ALUVIA®; lopinavir and ritonavir); TRIUMEQ@ (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; cabotegravir and rilpivirine; cabotegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir and lamivudine; lamivudine, abacavir and zidovudine; lamivudine and abacavir; lamivudine and tenofovir disoproxil fumarate; lamivudine, zidovudine and nevirapine; lopinavir and ritonavir; lopinavir, ritonavir, abacavir and lamivudine; lopinavir, ritonavir, zidovudine, and lamivudine; tenofovir and lamivudine; tenofovir disoproxil fumarate, emtricitabine and rilpivirine hydrochloride; lopinavir, ritonavir, zidovudine and lamivudine; Vacc-4× and romidepsin; and APH-0812.

### Other HIV Drugs

Examples of other drugs for treating HIV include acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, ABX-464, AG-1105, APH-0812, BIT-225, CYT-107, HGTV-43, HPH-16, HS-10234, IMO-3100, IND-02, MK-1376, MK-8507, MK-8591 November-205, PA-1050040 (PA-040), PGN-007, SCY-635, SB-9200, SCB-719, TR-452, TEV-90110, TEV-90112, TEV-90111, TEV-90113, RN-18, Immuglo, and VIR 576.

### HIV Nucleoside Reverse Transcriptase Translocation Inhibitors

Examples of HIV nucleoside reverse transcriptase translocation inhibitors (“NRTTIs”) include 4′-Ethynyl-2-fluoro-2′-deoxyadenosine triphosphate (also known as MK-8591 and EFdA).

### HIV Protease Inhibitors

Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG 17, TMB-657 (PPL-100), T-169, BL-008, and TMC-310911.

### HIV Reverse Transcriptase Inhibitors

Examples of HIV non-nucleoside or non-nucleotide inhibitors of reverse transcriptase include dapivirine, delavirdine, delavirdine mesylate, efavirenz, etravirine, lentinan, nevirapine, rilpivirine, AIC-292, KM-023, and VM-1500. Further examples of non-nucleoside reverse transcriptase inhibitors are disclosed in U.S. Patent Publication No. US2016/0250215. HIV nucleoside or nucleotide inhibitors of reverse transcriptase

Examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX@ and VIDEX EC@ (didanosine, ddl), abacavir, abacavir sulfate, alovudine, apricitabine, censavudine, didanosine, elvucitabine, festinavir, fosalvudine tidoxil, CMX 157, dapivirine, etravirine, OCR-5753, tenofovir disoproxil orotate, fozivudine tidoxil, lamivudine, phosphazid, stavudine, zalcitabine, zidovudine, GS-9131, GS-9148, KP-1461, and 4′-ethynyl-2-fluoro-2′-deoxyadenosine (EFdA).

### HIV Integrase Inhibitors

Examples of HIV integrase inhibitors include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, diketo quinolin-4-1 derivatives, integrase-LEDGF inhibitor, ledgins, M-522, M-532, NSC-310217, NSC-371056, NSC 48240, NSC-642710, NSC-699171, NSC-699172, NSC-699173, NSC-699174, stilbenedisulfonic acid, T-169 and cabotegravir.

Examples of HIV non-catalytic site, or allosteric, integrase inhibitors (NCINI) include CX-05045, CX-05168, and CX-14442.

### HIV Entry Inhibitors

Examples of HIV entry (fusion) inhibitors include cenicriviroc, CCR5 inhibitors, gp41 inhibitors, CD4 attachment inhibitors, gp120 inhibitors, and CXCR4 inhibitors.

Examples of CCR5 inhibitors include aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

Examples of gp41 inhibitors include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

Examples of CD4 attachment inhibitors include ibalizumab and CADA analogs.

Examples of gp120 inhibitors include Radha-108 (receptol) 3B3-PE38, BanLec, bentonite-based nanomedicine, fostemsavir tromethamine, IQP-0831, and BMS-663068.

Examples of CXCR4 inhibitors include plerixafor, ALT-1188, N15 peptide, and vMIP (Haimipu)

### HIV Maturation Inhibitors

Examples of HIV maturation inhibitors include BMS-955176 and GSK-2838232.

### Latency Reversing Agents

Examples of latency reversing agents include histone deacetylase (HDAC) inhibitors, proteasome inhibitors such as velcade, protein kinase C (PKC) activators, BET-bromodomain 4 (BRD4) inhibitors, ionomycin, PMA, SAHA (suberanilohydroxamic acid, or suberoyl, anilide, and hydroxamic acid), IL-15, JQ1, disulfram, amphotericin B, and ubiquitin inhibitors such as largazole analogs, and GSK 343.

Examples of HDAC inhibitors include romidepsin, vorinostat, and panobinostat.

Examples of PKC activators include indolactam, prostratin, ingenol B, and DAG lactones.

### Capsid Inhibitors

Examples of capsid inhibitors include capsid polymerization inhibitors or capsid disrupting compounds, HIV nucleocapsid p7 (NCp7) inhibitors such as azodicarbonamide, HIV p24 capsid protein inhibitors, AVI-621. AVI-101, AVI-201, AVI-301, and AVI-CANI-15 series.

### Immune-Based Therapies

Examples of immune-based therapies include toll-like receptors modulators such as tlr1, tlr2, tlr3, tlr4, tlr5, tlr6, dr7, dr8, dr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; IL-12; WF-10; VGV-1; MOR-22; BMS-936559; CYT 107, interleukin-IS/Fc fusion protein, normferon, peginterferon alfa-2a, peginterferon alfa-2b, recombinant interleukin-15, RPI-MN, GS-9620, and IR-103.

### Phosphatidylinositol3-Kinase (PI3K) Inhibitors

Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD 8186, BAY-1082439, CLR-1401, CLR-457, CUDC-907, DS-7423, EN-3342, GSK 2126458, GSK-2269577, GSK-2636771, INCB-040093, LY-3023414, MLN-1117, PQR 309, RG-7666, RP-6530, RV-1729, SAR-245409, SAR-260301, SF-i126, TGR-1202, UCB-5857, VS-5584, XL-765, and ZSTK-474.

### HIV Antibodies, Bispecific Antibodies, and “Antibody-Like” Therapeutic Proteins

Examples of HIV antibodies, bispecific antibodies, and “antibody-like” therapeutic proteins include DARTs@, DUOBODIES@, BITES®, XmAbs@, TandAbs@, Fab derivatives, bnABs (broadly neutralizing HIV-1 antibodies), BMS-936559, TMB 360, and those targeting HIV gp120 or gp41, antibody-Recruiting Molecules targeting HIV, anti-CD63 monoclonal antibodies, anti-GB virus C antibodies, anti-GP120/CD4, CCR5 bispecific antibodies, anti-nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, and Immuglo, and MB-66. Examples of those targeting HIV in such a manner include bavituximab, UB-421, C2F5, C2G12, C4E10, C2FS+C2G12+C4E10, 3-BNC-117, PGT145, PGT121, MDX010 (ipilimumab), VRC01, A32, 7B2, 10E8, VRC-07-523, VRC-HIVMAB080-00-AB, MGD-014, and VRC07.

### Pharmacokinetic Enhancers

Examples of pharmacokinetic enhancers include cobicistat and ritonavir.

### More Additional Therapeutic Agents

Examples of more additional therapeutic agents include the compounds disclosed in WO 2004/096286, WO 2006/015261, WO 2006/110157, WO 2012/003497, WO 2012/003498, WO 2012/145728, WO 2013/006738, WO 2013/159064, WO 2014/100323, US 2013/0165489, US 2014/0221378, US 2014/0221380, WO 2009/062285, WO 2010/130034, WO 2013/006792, US20140221356, US20100143301, and WO 2013/091096.

### HIV Vaccines

Examples of HIV vaccines include peptide vaccines, recombinant subunit protein vaccines, live vector vaccines, DNA vaccines, CD4-derived peptide vaccines, vaccine combinations, rgp120 (AIDSVAX), ALVAC HIV (vCP1521)/AIDSVAX B/E (gp120) (RV144), monomeric gp 120 HIV-1 subtype C vaccine, Remune, ITV-1, Contre Vir, Ad5 ENVA-48, DCVax-001 (CDX-2401), Vacc-4x, Vacc-C5, VAC-3S, multiclade DNA recombinant adenovirus-S(rAd5), Pennvax-G, Pennvax-GP, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF 59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV PT123, rAAV1-PG9DP, GOVX-B11, GOVX-B21, TVI-HIV-1, Ad-4 (Ad4-env Clade C+Ad4-mGag), EN41-UGR7C, EN41-FPA2, PreVaxTat, AE-H. MYM-V101, CombiHIVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, AGS-004, AVX-101, AVX-201, PEP-6409, SAV-001, Th V-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine. MVA vaccine, Pennvax-GP, pp71-deficient HCMV vector HIV gag vaccine, recombinant peptide vaccine (HIV infection), NCI, rgp160 HIV vaccine, RNActive HIV vaccine, SCB-703, Tat Oyi vaccine, TBC-M4, therapeutic HIV vaccine, UBI HIV gp120. Vacc-4x+romidepsin, variant gp 120 polypeptide vaccine, and rAd5 gag-pol env A/B/C vaccine.

In some embodiments, kits comprising a pharmaceutical composition of the invention disclosed herein in combination with one or more (e.g., one, two, three, one or two, or one to three) additional therapeutic agents are provided.

### EMBODIMENTS

The invention is further described by the embodiments set forth below.

In some embodiments, the invention is fixed dose combination for oral administration comprising: (a) from about 25% to about 80% of the weight of the tablet (w/w), a doravirine amorphous solid dispersion comprising doravirine and a polymer; and (b) from about 0.01% to about 0.12% w/w of islatravir.

In some embodiments, the fixed dose combination is a tablet, a capsule, a caplet, or granules/powder within a pre-measured package.

In some embodiments, the fixed dose combination is a tablet.

In some embodiments, the granules/powder of the fixed dose combination is provided as minitablets or pellets.

In some embodiments, in the fixed dose combination, the weight ratio of doravirine to the polymer in the solid dispersion is about 1:3 to about 1:5.

In some embodiments, in the fixed dose combination, the weight ratio of doravirine to the polymer in the solid dispersion is about 1:4.

In some embodiments, in the fixed dose combination, the islatravir is islatravir monohydrate.

In some embodiments, in the fixed dose combination, the islatravir is one of anhydrate forms I, II, III, and IV.

In some embodiments, in the fixed dose combination, the islatravir is anhydrate form IV.

In some embodiments, the fixed dose combination further comprises
(c) from about 7.0% to about 25% w/w of a brittle diluent;
(d) from about 7.0% to about 25% w/w of a ductile diluent;
(e) from about 4.0% to about 15% w/w of a disintegrant;
(f) from about 0.2% to about 1.0% w/w of a glidant; and
(g) from about 0.1% to about 1.5% w/w of a lubricant;
   wherein the brittle diluent and the ductile diluent combined comprise about 14% to about 45% of the weight of the fixed dose combination.

In some embodiments, in the fixed dose combination, the brittle diluent is lactose monohydrate.

In some embodiments, in the fixed dose combination, the ductile diluent is microcrystalline cellulose.

In some embodiments, in the fixed dose combination, the brittle diluent is lactose monohydrate and the ductile diluent is microcrystalline cellulose.

In some embodiments, the fixed dose combination is a tablet, and the weight of the tablet is about 600 to about 1100 mg.

In some embodiments, in the fixed dose combination, the polymer is selected from the group consisting of hydroxypropyl methyl cellulose acetate succinate (HPMCAS), hydroxpropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, and poly vinylpyrrolidinone-polyvinylacetate copolymers.

In some embodiments, in the fixed dose combination, the polymer is HPMCAS.

In some embodiments, in the fixed dose combination, the disintegrant is croscarmellose sodium.

In some embodiments, in the fixed dose combination, the the glidant is colloidal silicon dioxide

In some embodiments, in the fixed dose combination, the lubricant is magnesium sterate.

In some embodiments, the fixed dose combination, comprises from about 9% to about 19% w/w of each of lactose monohydrate and microcrystalline cellulose, wherein the lactose monohydrate and microcrystalline cellulose combined comprise from about 25% to about 31% of the weight of the fixed dose combination.

In some embodiments, in the fixed dose combination, each of lactose monohydrate and microcrystalline cellulose is present at from about 13% to about 15% of the weight of the fixed dose combination.

In some embodiments, in the fixed dose combination, the doravirine amorphous solid dispersion comprises from about 45% to about 80% of the weight of the fixed dose combination.

In some embodiments, in the fixed dose combination, the doravirine amorphous solid dispersion comprises from about 50% to about 70% of the weight of the fixed dose combination

In some embodiments, in the fixed dose combination, islatravir comprises from about 0.02% to about 0.08% of the weight of the fixed dose combination.

In some embodiments, in the fixed dose combination, islatravir comprises from about 0.02% to about 0.05% of the weight of the fixed dose combination.

In some embodiments, the fixed dose combination is a tablet weighing from about 650 mg to about 1000 mg.

In some embodiments, the fixed dose combination is a tablet weighing from about 700 mg to about 800 mg.

In some embodiments, the the fixed dose combination comprises:
(a) about 65% w/w of a solid dispersion comprising amorphous doravirine dispersed within a matrix formed by a polymer, wherein the polymer is HPMCAS and the weight ratio of doravirine to HPMCAS in the solid dispersion is about 1:4;
(b) about 0.0325% w/w of islatravir;
(c) about 12.48% w/w of lactose monohydrate;
(d) about 12.48% w/w of microcrystalline cellulose;
(e) about 9.0% w/w of croscarmellose sodium as the disintegrant;
(f) about 0.5% w/w of colloidal silicon dioxide as the glidant; and
(g) about 0.5% w/w of magnesium stearate as the lubricant.

In some embodiments, the the fixed dose combination is a tablet comprising:
(a) about 100 mg of the doravirine amorphous solid dispersion, wherein the polymer is HPMCAS and the weight ratio of doravirine to HPMCAS in the solid dispersion is about 1:4;
(b) about 0.25 mg of islatravir;
(c) about 96 mg of lactose monohydrate;
(d) about 96 mg of microcrystalline cellulose;
(e) about 69.2 mg of croscarmellose sodium;
(f) about 3.8 mg of colloidal silica; and
(g) about 3.8 mg of magnesium stearate, wherein the tablet weighs about 769 mg.

In some embodiments, the fixed dose combination further comprises a film coat, the film coat comprising:
(a) a film former selected from the group consisting of hydroxypropyl methy cellulose (Hypromellose, HPMC), hydroxy propyl cellulose (HPC), polyvinyl alcohol, and polyethylene glycol-poly vinyl alcohol (PEG-PVA) graft copolymer;
(b) a component selected from the group consisting of lactose monohydrate, mannitol, polydextrose, and isomalt; and
(c) a plasticizer selected from the group consisting of triacetin, polyethylene glycol, and a medium chain triglyceride; and
(d) an opacifier selected from the group consisting of calcium carbonate, rice starch, and titanium dioxide.

In some embodiments, the film coat of the fixed dose combination comprises:
a first film coat comprising hypromellose, lactose monohydrate, triacetin, and either calcium carbonate or titanium dioxide at about 20-50%, about 10-25%, about 5-15%, and about 20-37.5% w/w, respectively, of the first film coat; and, optionally,
a second film coat underneath the first film coat, wherein the second film coat comprises hypermellose and triacetin in the ratio of about 88-93% to about 7.5-12%, respectively;
wherein the first and the second filmcoats lead to about 3-7% and about 0.5-3% w/w weight gain, respectively, of the fixed dose combination.

In some embodiments, in the fixed dose combination, the first film coat comprises hypromellose, lactose monohydrate, triacetin, calcium carbonate, and colorants at about 35%, about 20%, about 10%, about 30-40%, and about 0-10%, respectively, of the weight of first film coat.

In some embodiments, in the fixed dose combination, the first film coat comprises hypromellose, lactose monohydrate, triacetin, titanium dioxide, and colorants at about 35%, about 20%, about 10%, and about 20-30%, and about 0-10%, respectively, of the weight of first film coat

In some embodiments, in the fixed dose combination, the second film coat comprises hypermellose and triacetin at a ratio of about 91% to 9%.

In some embodiments, in fixed dose combination, the film coat leads to about 3-8% weight gain of the fixed dose combination.

In some embodiments, the fixed dose combination further comprises a polishing agent.

In some embodiments, in fixed dose combination, the polishing agent is carnauba wax.

In some embodiments, the fixed dose combination is a tablet having length of about 15 mm to about 21 mm and breadth of about 7.5 mm to about 10.5 mm.

In some embodiments, the fixed dose combination is a tablet fixed dose combination length of about 17.5 mm and breadth of about 8.7 mm.

In some embodiments, the fixed dose combination has an in vitro dissolution rate of 80% release in 45 minutes in phosphate buffer containing Tween 80.

In some embodiments, provided herein is a method of treating of HIV infection in a subject in need thereof, the method comprising administering the fixed dose combination of any of the above embodiments to the subject. In some embodiments, in this method, the fixed dose combination is administered in a daily dosing regimen. In some embodiments, in this method, the fixed dose combination is administered once daily.

In some embodiments, provided herein is method of treating of HIV infection in a subject in need thereof, the method comprising administering to the subject the tablet comprising: a) about 100 mg of the doravirine amorphous solid dispersion, wherein the polymer is HPMCAS and the weight ratio of doravirine to HPMCAS in the solid dispersion is about 1:4; b) about 0.25 mg of islatravir; c) about 96 mg of lactose monohydrate; d) about 96 mg of microcrystalline cellulose; e) about 69.2 mg of croscarmellose sodium; f) about 3.8 mg of colloidal silica; and g) about 3.8 mg of magnesium stearate, wherein the tablet weighs about 769 mg. In some embodiments, in this method, the tablet is administered in a daily dosing regimen. In some embodiments, in this method, the tablet is administered once daily.

In some embodiments, provided herein is a method of treating of HIV infection in a subject in need thereof, the method comprising administering the fixed dose combination of any of the above embodiments to the subject in combination with an additional therapeutic agent. In some embodiments, in this method, the additional therapeutic agent is selected from the group consisting of: a combination drug for treating HIV (HIV combination drug), other drug for treating HIV, an HIV protease inhibitor, an HIV non-nucleoside or non-nucleotide inhibitor of reverse transcriptase, an HIV nucleoside or nucleotide inhibitor of reverse transcriptase, an HIV integrase inhibitor, an HIV non-catalytic site (or allosteric) integrase inhibitor, an HIV entry inhibitor, an HIV maturation inhibitor, a latency reversing agent, a compound that targets the HIV capsid, an immune-based therapy, a phosphatidylinositol 3 kinase (PI3K) inhibitor, an HIV antibody, a bispecific antibody and an “antibody-like” therapeutic protein, an HIV p17 matrix protein inhibitor, an IL-13 antagonist, a peptidyl-prolyl cis-trans isomerase A modulator, a protein disulfide isomerase inhibitor. a complement C5a receptor antagonist, a DNA methyltransferase inhibitor, an HIV vif gene modulator, a Vif dimerization antagonist, an HIV-1 viral infectivity factor inhibitor, a TAT protein inhibitor, an HIV-1 Nef modulator, an Hck tyrosine kinase modulator, a mixed lineage kinase-3 (MLK 3) inhibitor, an HIV-1 splicing inhibitor, a Rev protein inhibitor, an integrin antagonist, a nucleoprotein inhibitor, a splicing factor modulator, a COMM domain containing protein 1 modulator, an HIV ribonuclease H inhibitor, a retrocyclin modulator, a CDK-9 inhibitor, a dendritic ICAM-3 grabbing nonintegrin 1 inhibitor, an HIV GAG protein inhibitor, an HIV POL protein inhibitor, a Complement Factor H modulator, a ubiquitin ligase inhibitor, a deoxycytidine kinase inhibitor, a cyclin dependent kinase inhibitor, a proprotein convertase PC9 stimulator, an ATP dependent RNA helicase DDX3X inhibitor, a reverse transcriptase priming complex inhibitor, a G6PD and NADH-oxidase inhibitor, a pharmacokinetic enhancer, an HIV gene therapy agent, an HIV vaccine, an HIV nucleoside reverse transcriptase translocation inhibitor, an immunomodulator, an immunotherapeutic agent, an antibody-drug conjugate, a gene modifier, a gene editor (e.g., CRISPR/Cas9, zinc finger nucleases, homing nucleases, synthetic nucleases, TALENs), a cell therapy (e.g., chimeric antigen receptor T cell (e.g., YESCARTA@ (axicabtagene ciloleucel), engineered T cell receptors, and TCR-T) and a combination thereof.

In some embodiments, provided herein is any of the fixed dose combinations described herein, for use in treating an HIV infection. In some embodiments, in this method, the fixed dose combination is for administering in a daily dosing regimen. In some embodiments, in this method, the fixed dose combination is for administering in a once daily dosing regimen.

In some embodiments, provided herein is a tablet for use in treating an HIV infection, the tablet comprising: a) about 100 mg of the doravirine amorphous solid dispersion, wherein the polymer is HPMCAS and the weight ratio of doravirine to HPMCAS in the solid dispersion is about 1:4; b) about 0.25 mg of islatravir; c) about 96 mg of lactose monohydrate; d) about 96 mg of microcrystalline cellulose; e) about 69.2 mg of croscarmellose sodium; f) about 3.8 mg of colloidal silica; and g) about 3.8 mg of magnesium stearate, wherein the tablet weighs about 769 mg, and wherein the tablet is administered in a once daily dosing regimen.

In some embodiments, provided herein is a use of the fixed dose combination of any of of the embodiments described herein in the manufacture of a medicament for treating an HIV infection. In some embodiments, in this method, the fixed dose combination is administered in a daily dosing regimen. In some embodiments, in this method, the fixed dose combination is administered in a once daily dosing regimen.

In some embodiments, provided herein is a use of a tablet in the manufacture of a medicament for treating an HIV infection, the tablet comprising: a) about 100 mg of the doravirine amorphous solid dispersion, wherein the polymer is HPMCAS and the weight ratio of doravirine to HPMCAS in the solid dispersion is about 1:4; b) about 0.25 mg of islatravir; c) about 96 mg of lactose monohydrate; d) about 96 mg of microcrystalline cellulose; e) about 69.2 mg of croscarmellose sodium; f) about 3.8 mg of colloidal silica; and g) about 3.8 mg of magnesium stearate. In some embodiments, in this method, the fixed dose combination is administered in a daily dosing regimen. In some embodiments, in this method, the fixed dose combination is administered in a once daily dosing regimen.

In some embodiments, provided herein is a use of the fixed dose combination of any of of the embodiments described herein in the manufacture of a medicament for treating an HIV infection in combination with an additional therapeutic agent. In some embodiments, the additional therapeutic agent is selected from the group consisting of: a combination drug for treating HIV (HIV combination drug), other drug for treating HIV, an HIV protease inhibitor, an HIV non-nucleoside or non-nucleotide inhibitor of reverse transcriptase, an HIV nucleoside or nucleotide inhibitor of reverse transcriptase, an HIV integrase inhibitor, an HIV non-catalytic site (or allosteric) integrase inhibitor, an HIV entry inhibitor, an HIV maturation inhibitor, a latency reversing agent, a compound that targets the HIV capsid, an immune-based therapy, a phosphatidylinositol 3 kinase (PI3K) inhibitor, an HIV antibody, a bispecific antibody and an “antibody-like” therapeutic protein, an HIV p17 matrix protein inhibitor, an IL-13 antagonist, a peptidyl-prolyl cis-trans isomerase A modulator, a protein disulfide isomerase inhibitor, a complement C5a receptor antagonist. a DNA methyltransferase inhibitor, an HIV vif gene modulator, a Vif dimerization antagonist, an HIV-1 viral infectivity factor inhibitor, a TAT protein inhibitor, an HIV-1 Nef modulator, an Hck tyrosine kinase modulator, a mixed lineage kinase-3 (MLK 3) inhibitor, an HIV-1 splicing inhibitor, a Rev protein inhibitor, an integrin antagonist, a nucleoprotein inhibitor, a splicing factor modulator, a COMM domain containing protein 1 modulator, an HIV ribonuclease H inhibitor, a retrocyclin modulator, a CDK-9 inhibitor, a dendritic ICAM-3 grabbing nonintegrin 1 inhibitor, an HIV GAG protein inhibitor, an HIV POL protein inhibitor, a Complement Factor H modulator, a ubiquitin ligase inhibitor, a deoxycytidine kinase inhibitor, a cyclin dependent kinase inhibitor, a proprotein convertase PC9 stimulator, an ATP dependent RNA helicase DDX3X inhibitor, a reverse transcriptase priming complex inhibitor, a G6PD and NADH-oxidase inhibitor, a pharmacokinetic enhancer, an HIV gene therapy agent, an HIV vaccine, an HIV nucleoside reverse transcriptase translocation inhibitor, an immunomodulator, an immunotherapeutic agent, an antibody-drug conjugate, a gene modifier, a gene editor (e.g., CRISPR/Cas9, zinc finger nucleases, homing nucleases, synthetic nucleases, TALENs), a cell therapy (e.g., chimeric antigen receptor T cell (e.g., YESCARTA@ (axicabtagene ciloleucel), engineered T cell receptors, and TCR-T) and a combination thereof.

In some embodiments, provided herein is a pharmaceutical unit dose comprising the fixed dose combination of any of the above embodiments.

In some embodiments, provided herein is a pharmaceutical unit dose comprising a) about 100 mg of the doravirine amorphous solid dispersion, wherein the polymer is HPMCAS and the weight ratio of doravirine to HPMCAS in the solid dispersion is about 1:4; b) about 0.25 mg of islatravir; c) about 96 mg of lactose monohydrate; d) about 96 mg of microcrystalline cellulose; e) about 69.2 mg of croscarmellose sodium; f) about 3.8 mg of colloidal silica; and g) about 3.8 mg of magnesium stearate. In some embodiments, this pharmaceutical unit dose is for administering in a daily dosing regimen. In some embodiments, this pharmaceutical unit dose is for administering in a once daily dosing regimen.

In some embodiments, provided herein is a pharmaceutical unit dose comprising the fixed dose combination of any of the above embodiments having 100 mg of doravirine and 0.25 mg of islatravir. In some embodiments, this pharmaceutical unit dose is for administering in a daily dosing regimen. In some embodiments, this pharmaceutical unit dose is for administering in a once daily dosing regimen.

### EXEMPLIFICATION

The invention now being generally described, it will be more readily understood by reference to the following examples, which are included merely for purposes of illustration of certain embodiments of the present invention and are not intended to limit the invention. In this examples, Doravirine is spray dried as an amorphous spray dried intermediate (SDI) containing doravirine and hypermellose acetate succinate in the ratio of 1:4.

### Example 1: Tablet Formulations to Increase Doravirine SDI Loading while Balancing Tablet Integrity

Tablet formulations were studied for ability to be defect free when compressed at reasonable press speeds using a small scale compaction simulator. Tablet integrity was quantified by visual observations for defects such cracks on tablet surface on 10 tablets as a function of press speed. The simulated press speed at which defects were first noted were used as a metric for comparing the formulations.

Eight different formulations containing different levels of doravirine SDI with variations in the levels of common brittle diluents mannitol and lactose monohydrate, and the ductile diluent microcrystalline cellulose were studied as shown in Tables 1, 2 and 3. Tablets were made by blending excipients along with the SDI and slugging into granules prior to compression on a compaction simulator. A suitable oval or modified oval image was selected to study these formulations. Image size was appropriately selected for different tablet weights which change with the SDI loading in the product.

Composition of the formulations with 50% SDI loading studied are shown in Table 1. Formulation 1 contained 1:1 ratio of microcrystalline cellulose to lactose monohydrate (21.5% w/w each). Formulations 2 and 3 contained mannitol and lactose monohydrate, respectively, as the diluent at 42.5% (w/w). The results (Table 1) demonstrate that at 50% SDI loading, Formulation 3 provides better tablet integrity performance than a formulation containing only brittle diluents.

Formulations with 75% SDI loading were studied as shown in Table 2. Formulation 4 has 1:1 ratio of microcrystalline cellulose to lactose monohydrate, and Formulation 5 has only lactose monohydrate as diluent. Comparing the tablet integrity results between Formulation 1-5, it was observed that while 75% SDI loading formulation had a smaller tablet weight, the increase in SDI loading in the formulation reduced the speed at which failure of tablet integrity was observed.

Tablet integrity assessment was conducted at SDI loading of 65% by varying the level of microcrystalline cellulose and lactose monohydrate (Formulations 6, 7, and 8 with 1:1, 2:1 and 1:2 ratios respectively). The maximum speed at which no defect was observed for 10 samples was taken as the measure of tabet integrity. The results, shown in Table 3, indicate that tablet integrity was not sensitive to variations in the level of microcrystalline cellulose and lactose monohydrate that were examined.

TABLE 1
Core Tablet Formulations studies at 50% SDI loading and changing diluent levels

| | Formulation-1 | | Formulation-2 | | Formulation-3 | |
|---|---|---|---|---|---|---|
| | Amount | | Amount | | Amount | |
| Components | [mg] | w/w % | [mg] | w/w % | [mg] | w/w % |
| Doravirine Spray Dried | 500.00 | 50.00 | 500.00 | 50.00 | 500.00 | 50.00 |
| Intermediate | | | | | | |
| Microcrystalline Cellulose | 215.0 | 21.50 | — | — | — | — |
| Lactose Monohydrate | 215.0 | 21.50 | — | — | 425.0 | 42.5 |
| Mannitol | — | — | 425.0 | 42.5 | — | — |
| Croscarmellose Sodium | 30.00 | 3.000 | 30.00 | 3.000 | 30.00 | 3.000 |
| Colloidal Silica | 5.000 | 0.500 | 5.000 | 0.500 | 5.000 | 0.500 |
| Magnesium Stearate | 1.250 | 0.250 | 1.250 | 0.250 | 1.250 | 0.250 |

Extra Granular Components

| Croscarmellose Sodium | 30.00 | 3.000 | 30.00 | 3.000 | 30.00 | 3.000 |
|---|---|---|---|---|---|---|
| Magnesium Stearate | 1.250 | 0.250 | 1.250 | 0.250 | 1.250 | 0.250 |
| Core Tablet | 1000 | 100.0 | 1000 | 100.0 | 1000 | 100. |
| Simulated Press Speed at | 60 | | 15 | | 30 | |
| which tablet integrity starts to | | | | | | |
| fail (rpm) | | | | | | |

TABLE 2
Core Tablet Formulations studies at 75%
SDI loading and changing diluent levels

| | Formulation-4 | | Formulation-5 | |
|---|---|---|---|---|
| | Amount | | Amount | |
| Components | [mg] | w/w % | [mg] | w/w % |
| Doravirine Spray Dried | 500.00 | 75.00 | 500.00 | 75.00 |
| Intermediate¹ | | | | |
| Microcrystalline Cellulose | 50.00 | 7.50 | — | — |
| Lactose Monohydrate | 50.00 | 7.50 | 80.0 | 12.00 |
| Croscarmellose Sodium | 60.00 | 9.000 | 80.00 | 12.00 |
| Colloidal Silica | 3.333 | 0.500 | 3.333 | 0.500 |
| Magnesium Stearate | 1.67 | 0.250 | 1.67 | 0.250 |

Extra Granular Components

| | | | | |
|---|---|---|---|---|
| Magnesium Stearate | 1.67 | 0.250 | 1.67 | 0.250 |
| Core Tablet | 667 | 100.0 | 667 | 100.0 |
| Simulated Press Speed at | 40 | — | 30 | — |
| which tablet integrity | | | | |
| starts to fail (rpm) | | | | |

¹Doravirine spray dried intermediate contains doravirine and hypermellose acetate succinate in the ratio of 1:4.

TABLE 3
Core Tablet Formulations studies at 65% SDI loading and changing diluent levels

| | Formulation-6 | | Formulation-7 | | Formulation-8 | |
|---|---|---|---|---|---|---|
| | Amount | | Amount | | Amount | |
| Components | [mg] | w/w % | [mg] | w/w % | [mg] | w/w % |
| Doravirine Spray Dried | 500.00 | 65.00 | 500.00 | 65.00 | 500.00 | 65.00 |
| Intermediate¹ | | | | | | |
| Microcrystalline Cellulose | 107.7 | 14.00 | 143.6 | 18.67 | 71.77 | 9.33 |
| Lactose Monohydrate | 107.7 | 14.00 | 71.77 | 9.33 | 143.6 | 18.67 |
| Croscarmellose Sodium | 46.15 | 6.000 | 46.15 | 6.000 | 46.15 | 6.000 |
| Colloidal Silica | 3.846 | 0.500 | 3.846 | 0.500 | 3.846 | 0.500 |
| Magnesium Stearate | 1.923 | 0.250 | 1.923 | 0.250 | 1.923 | 0.250 |

Extra Granular Components

| Magnesium Stearate | 1.923 | 0.250 | 1.923 | 0.250 | 1.923 | 0.250 |
|---|---|---|---|---|---|---|
| Core Tablet | 769 | 100.0 | 769 | 100.0 | 769 | 100.0 |
| Maximum Press speed | 50 | | 50 | | 50 | |
| (simulated)studied at which | | | | | | |
| no tablet defects occur (rpm) | | | | | | |

### Example 2: Image Shape to Improve Tablet Integrity

Three tooling designs of varying dimensions were evaluated for ability to produce defect free tablets. All evaluations utilized the composition comprising Islatravir Formulation 9 in Table 4. The excipients, islatravir and the doravirine spray dried intermediate were blended in a blender, comilled, and then blended again. The resulting blend was lubricated, roller compacted, milled, and then lubricated after granulation. The tableting at simulated press speeds was conducted on a compaction simulator. Visual assessment was conducted to confirm the absence of defects on 10 tablets. Results demonstrate better performance using tooling design 3.

TABLE 4
Doravirine/Islatravir Formulation with 65% SDI loading

Formulation-9

| | Amount | |
|---|---|---|
| Components | [mg] | w/w % |
| Doravirine Spray Dried Intermediate¹ | 500.00 | 65.00 |
| Islatravir | 2.25 | 0.293 |
| Microcrystalline Cellulose | 95.02 | 12.35 |
| Lactose Monohydrate | 95.02 | 12.35 |
| Croscarmellose Sodium | 69.23 | 9.000 |
| Colloidal Silica | 3.846 | 0.500 |
| Magnesium Stearate | 1.923 | 0.250 |

Extra Granular Components

| | | |
|---|---|---|
| Magnesium Stearate | 1.923 | 0.250 |
| Core Tablet | 769.2 | 100.0 |

TABLE 5
Impact of tablet design on tablet integrity for Formulation 9

| Attributes | Tooling #1 | Tooling #2 | Tooling #3 |
|---|---|---|---|
| Shape | Modified capsule | | |
| Length | 16.66 | 18.29 | 17.48 |
| Breadth | 8.33 | 9.14 | 8.74 |
| Cup depth | 1.02 | 1.12 | 1.07 |
| Taper | Yes | Yes | Yes |
| Maximum Press speed | 30 | 30 | 50 |
| (simulated) studied at which | | | |
| no tablet defects occur (rpm) | | | |

### Example 3: Improvement of Content Uniformity of Islatravir in Low-Dose Islatravir Drug Product Formulations

The dose of islatravir in the Doravirine/Islatravir fixed dose combination tablet is 0.25 mg. At such low doses, islatravir drug loading is about 0.02-0.04% of the weight of tablet. Therefore, there is a risk of failing content uniformity with respect to isltravir. On the other hand, doravirine, with higher drug loading, has a low risk of failing content uniformity in the drug product.

Preliminary solubilization studies with mixtures of islatravir, various excipients, and doravirine spray dried intermediate showed that islatravir did not fully dissolve unless the polymer was fully dissolved indicating a propensity of islatravir to adhere to the polymer. The islatravir/doravirine formulation (Formulations 10, 11) contains SDI, which includes the polymer hypromellose acetate succinate and other diluents as shown in Table 6 below. The manufacturing process includes standard unit operations and equipment for tablet production. The core tablet is prepared by (i) optional de-agglomeration while dispensing islatravir, excipients, and doravirine SDI and blending these components in a blender for 250 revolutions, (ii) comilling using a 610 μm or appropriate screen (iii) blending for 250 revolutions and lubrication of the blend with magnesium stearate screened through 250 μm screen for 125 revolutions, (iv) roller compaction using Alexanderwerk WP-200 at roll force and roll gap of 4.8 kN/cm and 2 mm for Formulation 10A, 6 kN/cm and 3 mm for Formulation 10B, and 8.3 kN/cm, 2 mm for Formulation 11, (v) lubrication of roller compacted blend with magnesium stearate screened through 250 μm screen for 125 revolutions, (vi) compression of tablets using an appropriate tooling (17.48 mm×8.74 mm for Formulation 10A, 18.29 mm×9.14 mm oval image for Formulation 10B and 19.0 mm×19.5 mm oval image for Formulation 11) on a rotary tablet press, Fette 3090 such that the tablet weight is within +/−3% of the core tablet weight shown in Table 6. This formulation and process provides acceptable uniformity of islatravir in the core tablet despite the extremely low drug loading as shown in Table 6. The formulations also showed robust tableting performance without risk of cracking on rotary tablet press.

TABLE 6
Doravirine/Islatravir fixed dose formulation containing SDI at 65% and 50% w/w

| | Formulation 10 | | | | | |
|---|---|---|---|---|---|---|
| | A | | B | | Formulation 11 | |
| | Amount | | Amount | | Amount | |
| Components | [mg] | % w/w | [mg] | % w/w | [mg] | % w/w |
| Doravirine Spray dried | 500.00 | 65.00 | 500.00 | 65.00 | 500.00 | 50.00 |
| intermediate | | | | | | |
| Islatravir | 0.2653¹ | 0.0345 | 0.2500² | 0.0325 | 0.2500² | 0.0325 |
| Microcrystalline | 96.02 | 12.48 | 96.03 | 12.48 | 214.8 | 21.48 |
| Cellulose | | | | | | |
| Lactose Monohydrate | 96.02 | 12.48 | 96.03 | 12.48 | 214.8 | 21.48 |
| Croscarmellose | 69.23 | 9.000 | 69.23 | 9.000 | 60.00 | 6.000 |
| Sodium | | | | | | |
| Colloidal Silica | 3.846 | 0.500 | 3.846 | 0.500 | 2.500 | 0.500 |
| Magnesium Stearate | 3.846 | 0.500 | 3.846 | 0.500 | 2.500 | 0.500 |
| Core Tablet | 769.2 | 100.0 | 769.2 | 100.0 | 1000.0 | 100.0 |
| Average Label Claim | 98.5 | | 98.5 | | 98.2 | |
| during Content | | | | | | |
| Uniformity testing for | | | | | | |
| Islatravir (%) | | | | | | |
| Acceptance Value | 4.8 | | 2.4 | | 2.3 | |
| during Content | | | | | | |
| Uniformity testing for | | | | | | |
| Islatravir | | | | | | |

¹1.061 mg of Islatravir monohydrate is equivalent to 1.000 mg of Islatravir free form.
²1.000 mg of Islatravir anhydrate form IV is equivalent to 1.000 mg of Islatravir free form.

### Example 4: Filmcoat Selection For The Doravirine/Islatravir Drug Product

Filmcoats were studied for developing a film-coated Doravirine/Islatravir drug product with acceptable critical quality attributes. Three different filmcoats (Filmcoats 1, 2, and 3) with titanium dioxide as opacifier were considered. Filmcoat 1, at filmcoat weight gain of 3.0% w/w of the core tablet, showed acceptable stability and acceptable photostability of doravirine.

Titanium dioxide free filmcoats were studied by assessing different opacifiers such as calcium carbonate (Filmcoat 4) and rice starch (Filmcoats 5 and 6) at 3-7% filmcoat weight gains. Tablets having the starch based Filmcoat 5 was found to be at risk of photostability related degradation at 30° C./50% RH when exposed to 40 hours of 10W/hr UV light. Tablets with starch based Filmcoat 6 were found to be prone to spotted appearance at accelerated stability conditions. Tablets with calcium carbonate based filmcoat (Filmcoat 4 only) showed low extent for basic chemical degradation of islatravir despite the presence of basic calcium carbonate in the filmcoat. Tablets with dual coating, Filmcoat 7 as sub-coat and Filmcoat 4 as top coat was also studied. These tablets showed acceptable filmcoat appearance and not lack any critical quality attributes.

Carnauba wax was added as polishing agent (either incorporated into the coating composition or separately after filmcoating).

TABLE 7
Composition of titanium dioxide containing and titanium dioxide free filmcoats

| Film coat | Filmcoat | Filmcoat | Filmcoat | Filmcoat | Filmcoat | Filmcoat | Filmcoat |
|---|---|---|---|---|---|---|---|
| composition | 1 (% w/w) | 2 (% w/w) | 3 (% w/w) | 4 (% w/w) | 5 (% w/w) | 6 (% w/w) | 7 (% w/w) |
| Hydroxypropyl | 40.00 | | | 30.00 | 30.00 | 19.00 | 90.91 |
| methyl cellulose 2910 | | | | | | | |
| (6 cps) | | | | | | | |
| Hydroxypropyl | 5.000 | | | 5.000 | 5.000 | 13.00 | |
| methyl cellulose 2910 | | | | | | | |
| (15 cps) | | | | | | | |
| Polyvinyl alcohol | | 40.00 | 3.500 | | | | |
| Macrogol polyvinyl | | | 40.00 | | | | |
| alcohol graft | | | | | | | |
| copolymer | | | | | | | |
| Rice starch | | | | | 34.88 | 34.87 | |
| Isomalt | | | | | | 30.00 | |
| Medium chain | | | | | | 3.000 | |
| triglycerides | | | | | | | |
| GMCC Type 1 | | | 4.000 | | | | |
| mono/diglycerides | | | | | | | |
| Titanium Dioxide | 24.39 | 25.00 | 10.00 | | | | |
| Calcium Carbonate | | | | 34.88 | | | |
| Lactose monohydrate | 22.00 | | | 20.00 | 20.00 | | |
| Triacetin | 8.000 | | | 10.00 | 10.00 | | 9.090 |
| Polyethylene glycol | | 20.20 | | | | | |
| 3350 | | | | | | | |
| Talc | | 14.80 | 27.50 | | | | |
| Colorants | 0.570 | | 15.00 | 0.120 | 0.120 | 0.130 | |

### Example 5: Bioperformance of the Doravirine/Islatravir Fixed Dose Combination Drug Product

Doravirine/Islatravir Formulations with compositions shown in table 9 were studied in a relative bioavailability study to compare the performance against single entity formulations of doravirine and islatravir.

TABLE 8
Doravirine/Islatravir fixed dose formulations and single entity
formulatios used in the relative bioavailability study

| | | | | | Doravirine | Islatravir |
|---|---|---|---|---|---|---|
| | | | | | Single | Single |
| | | | | | Entity | Entity |
| | Formulation 12 | | Formulation 13 | | formulation | formulation |
| | Amount | % w/w (of | Amount | % w/w (of | Amount | Amount |
| Components | [mg] | core tablet) | [mg] | core tablet) | [mg] | [mg] |
| Doravirine Spray | 500.00 | 65.00 | 500.00 | 75.00 | 500.00 | 50.00 |
| dried intermediate | | | | | | |
| Islatravir | 0.7960¹ | 0.103 | 0.7960¹ | 0.119 | — | 0.7958¹ |
| Microcrystalline | 95.76 | 12.48 | 49.61 | 7.441 | — | 28.64 |
| Cellulose | | | | | | |
| Lactose | 95.76 | 12.48 | 49.61 | 7.441 | — | — |
| Monohydrate | | | | | | |
| Mannitol | — | — | — | — | — | 114.6 |
| Croscarmellose | 69.23 | 9.000 | 60.00 | 9.000 | 60.00 | 4.500 |
| Sodium | | | | | | |
| Colloidal Silica | 3.846 | 0.500 | 3.334 | 0.500 | 5.000 | |
| Magnesium | 3.846 | 0.500 | 3.334 | 0.500 | 5.000 | 1.500 |
| Stearate | | | | | | |
| Capsule | | | | | | Size #3, |
| | | | | | | white hard |
| | | | | | | gelatin |
| | | | | | | capsule |
| Core | 769.2 | 100.0 | 666.7 | 100.0 | 1000 | 150.0 |
| Tablet/Capsule Fill | | | | | | |
| Weight | | | | | | |
| Filmcoat Blend² | 23.08 | 3.000 | 20.00 | 3.000 | 30.00 | |
| Total filmcoated | 792.3 | 103.0 | 686.7 | 103.0 | 1030 | |
| tablet | | | | | | |

¹1.061 mg of Islatravir monohydrate is equivalent to 1.000 mg of Islatravir free form.
²Filmcoat blend contains hypermellose, triacetin, lactose, titanium dioxide, and colorants

The pharmacokinetic results show comparable performance of the fixed dose products at both 65% and 75% SDI loading formulations (when compared to single entity SE Doravirine formulation with 50% SDI loading, co-dosed with Islatravir single entity formulation) as shown in Table 9.

TABLE 9
Pharmacokinetics of Doravirine and Islatravir in Relative
Bioavailability Study Comparing Formulation 13 and
Formulation 12 against Co-administration of Doravirine
and Islatravir Single entity formulations

| | Doravirine | | Islatravir | |
|---|---|---|---|---|
| | GMR^{§} (90% CI^{∥}) | | GMR^{§} (90% CI) | |
| | Formulation | Formulation | Formulation | Formulation |
| | 13/SE^{¶} | 12/SE | 13/SE | 12/SE |
| AUC0-24 hr* | 1.08 (0.99, | 1.09 (1.01, | 1.02 (0.96, | 1.01 (0.97, |
| (nM(*hr) | 1.17) | 1.18) | 1.08) | 1.05) |
| Cmax^{†} (nM) | 1.09 (0.98, | 1.09 (0.98, | 0.91 (0.80, | 0.91 (0.81. |
| | 1.20) | 1.21) | 1.05) | 1.01) |
| C24^{‡} (nM) | 1.03 | 1.01 | N/A | N/A |

*AUC0-24 hr: area under the concentration - time curve from zero to 24-hour concentration
^{†}Cmax: maximum observed concentration
^{‡}C24: observed concentration at 24-hour time point.
^{§}Geometric Least-Square Mean Ratio
∥ CI = Confidence Interval
^{¶}SE = Co-administered single entities of islatravir (0.75 mg) and doravirine((100 mg) respectively

### Example 6: Dissolution Performance of the Doravirine/Islatravir Drug Product

Doravirine/Islatravir Formulation 12 having composition given in Table 10 was found to have different rates of dissolution introduced by process parameter variation and storage. The manufacturing process used for preparing the formulation includes (i) blending islatravir, the excipients, and doravirine spray dried intermediate, (ii) comilling, (iii) blending and lubrication, (iv) roller compaction and milling, (v) lubrication, (vi) compression and (vii) filmcoating and waxing. The reference formulation is packaged with desiccants in induction sealed high density polyethylene (HDPE) bottles. Test A formulation was prepared by equilibrating the reference formulation tablets under room temperature and target relative humidity of 45%, and then, packaging in sealed glass bottles. Test B formulation was manufactured with lower roller compaction force and compression force.

TABLE 10
Doravirine/Islatravir fixed dose formulation (Formulation
12) used in the relative bioavailability study

| | Amount | % w/w (of |
|---|---|---|
| Components | [mg] | core tablet) |
| Doravirine Spray dried intermediate | 500.00 | 65.00 |
| Islatravir | 0.7960¹ | 0.103 |
| Microcrystalline Cellulose | 95.76 | 12.45 |
| Lactose Monohydrate | 95.76 | 12.45 |
| Croscarmellose Sodium | 69.23 | 9.000 |
| Colloidal Silica | 3.846 | 0.500 |
| Magnesium Stearate | 3.846 | 0.500 |
| Core Tablet | 769.2 | 100.0 |
| Filmcoat Blend² | 23.08 | 3.000 |
| Carnauba wax | 0.0268 | 0.003 |
| Total filmcoated tablet | 792.3 | 103.0 |

¹1.061 mg of Islatravir monohydrate is equivalent to 1.000 mg of Islatravir free form.
²Filmcoat blend contains hypermellose, triacetin, lactose, titanium dioxide, and colorants

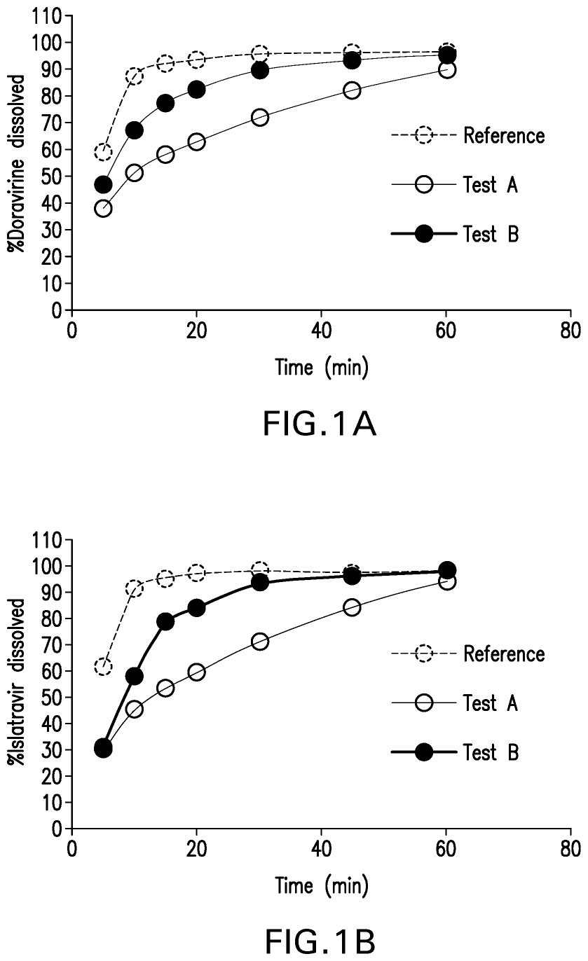

Dissolution was conducted in a USP II apparatus using 25 mM phosphate buffer (pH 6.8) containing 3% polysorbate 80. Results are shown in FIGS. 1A and 1B.

These formulations were studied in a relative bioavailability clinical study designed as a randomized, cross-over study. The pharmacokinetic comparison of doravirine and islatravir following single dose administration of doravirine/islatraavir FDC tablet (Reference, Test A, Test B) showed that Test A and Test B met bioequivalence criteria with 90% Cl for AUC, Cₘₐₓ, and C₂₄ as shown in Table 11. Dissolution of doravirine and islatravir corresponding to B formulation represents the safe space of acceptable dissolution performance of the doravirine-islatravir drug product.

TABLE 11
Pharmacokinetic Comparison of Doravirine and Islatravir in
the clinical formulations (Reference, Test A and Test B)

| | Doravirine | | Islatravir | |
|---|---|---|---|---|
| | Treatment | | | |
| | Test A/ | Test B/ | Test A/ | Test B/ |
| | Reference | Reference | Reference | Reference |
| Comparison | GMR^{§} (90% CI^{∥}) ^{a} | GMR^{§} (90% CI^{∥}) ^{a} | GMR^{§} (90% CI^{∥}) ^{a} | GMR^{§} (90% CI^{∥}) ^{a} |
| AUC_{0-∞} * | 1.01 (0.92, 1.10) | 1.05 (0.97, 1.14) | 1.00 (0.96, 1.05) | 1.00 (0.96, 1.04) |
| (nM)(*hr) | | | | |
| Cₘₐₓ^{†} (nM) | 1.07 (0.92, 1.24) | 1.08 (0.96, 1.21) | 1.05 (0.88, 1.26) | 0.97 (0.84. 1.12) |
| C₂₄^{‡} (nM) | 1.02 (0.93, 1.12) | 1.09 (1.00, 1.19) | 0.99 (0.95, 1.04) | 1.01 (0.97, 1.05) |

^{a} Back-transformed least-squares mean and confidence interval from linear mixed effects model performed on natural log-transformed values.
Reference = DOR/ISL FDC
Test A = DOR/ISL FDC, under alternate storage conditions
Test B = DOR/ISL FDC, manufactured with alternate conditions
FDC = fixed dose combination; DOR = doravirine; ISL = islatravir
* AUC0-24 hr: area under the concentration- time curve from zero to 24-hour concentration
^{†}Cmax: maximum observed concentration
^{‡}C24: observed concentration at 24-hour time point.
^{§}GMR Geometric Least-Square Mean Ratio
^{∥}CI = Confidence Interval

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Various structural elements of the different embodiments and various disclosed method steps may be utilized in various combinations and permutations, and all such variants are to be considered forms of the invention. Scope of the invention is thus indicated by the appended claims rather than by the foregoing description, and all changes that come within the meaning and range of equivalency of the claims are intended to be embraced therein.

1. A fixed dose combination for oral administration comprising:
a) from about 25% to about 80% by weight (w/w), a doravirine amorphous solid dispersion comprising doravirine and a polymer; and
b) from about 0.01% to about 0.12% w/w of islatravir monohydrate.

2. (canceled)

3. The fixed dose combination of claim 1, wherein the fixed dose combination is a tablet.

4. The fixed dose combination of claim 1, wherein the fixed dose combination is provided as granules/powder in a pre-measured package, and wherein the granules/powder is provided as minitablets or pellets.

5. The fixed dose combination of claim 1, wherein the weight ratio of doravirine to the polymer in the solid dispersion is about 1:4.

6-8. (canceled)

9. The fixed dose combination of claim 1, further comprising
c) from about 7.0% to about 25% w/w of a brittle diluent;
d) from about 7.0% to about 25% w/w of a ductile diluent;
e) from about 4% to about 15% w/w of a disintegrant;
f) from about 0.2% to about 1.0% w/w of a glidant; and
g) from about 0.1% to about 1.5% w/w of a lubricant;
wherein the combined weight of the brittle diluent and the ductile diluent comprise about 14% to about 45% of the weight of the fixed dose combination.

10-11. (canceled)

12. The fixed dose combination of claim 9, wherein the brittle diluent is lactose monohydrate and the ductile diluent is microcrystalline cellulose.

13. The fixed dose combination of claim 9, wherein the fixed dose combination is a tablet weighing about 600 to about 1100 mg.

14. The fixed dose combination of claim 1, wherein the polymer is selected from the group consisting of hydroxypropyl methyl cellulose acetate succinate (HPMCAS), hydroxpropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers.

15. The fixed dose combination of claim 14, wherein the polymer is HPMCAS.

16. The fixed dose combination of claim 9, wherein the disintegrant is croscarmellose sodium.

17. The fixed dose combination of claim 9, wherein the glidant is colloidal silicon dioxide.

18. The fixed dose combination of claim 9, wherein the lubricant is magnesium stearate.

19. The fixed dose combination of claim 9 comprising from about 9% to about 19% w/w of each of lactose monohydrate and microcrystalline cellulose, wherein the combined weight of the lactose monohydrate and the microcrystalline cellulose comprises from about 25% to about 31% of the weight of the fixed dose combination.

20. The fixed dose combination of claim 19, wherein each of the lactose monohydrate and the microcrystalline cellulose is present at from about 13% to about 15% of the weight of the fixed dose combination.

21. (canceled)

22. The fixed dose combination of claim 1, wherein the doravirine amorphous solid dispersion comprises from about 50% to about 70% of the weight of the fixed dose combination.

23. (canceled)

24. The fixed dose combination of claim 1, wherein the islatravir comprises from about 0.02% to about 0.05% of the weight of the fixed dose combination.

25. The fixed dose combination of claim 1, wherein the fixed dose combination is a tablet weighing from about 650 mg to about 1000 mg.

26. (canceled)

27. The fixed dose combination of claim 12 comprising:
a) about 65% w/w of a solid dispersion comprising amorphous doravirine dispersed within a matrix formed by a polymer, wherein the polymer is HPMCAS and the weight ratio of doravirine to HPMCAS in the solid dispersion is about 1:4;
b) about 0.0325% w/w of the islatravir;
c) about 12.48% w/w of the lactose monohydrate;
d) about 12.48% w/w of the microcrystalline cellulose;
e) about 9.0% w/w of croscarmellose sodium as the disintegrant;
f) about 0.5% w/w of colloidal silicon dioxide as the glidant; and
g) about 0.5% w/w of magnesium stearate as the lubricant.

28. The tablet of claim 13 comprising:
a) about 100 mg of the doravirine amorphous solid dispersion, wherein the polymer is HPMCAS and the weight ratio of doravirine to HPMCAS in the solid dispersion is about 1:4;
b) about 0.25 mg of the islatravir;
c) about 96 mg of the lactose monohydrate;
d) about 96 mg of the microcrystalline cellulose;
e) about 69.2 mg of croscarmellose sodium as the disintegrant;
f) about 3.8 mg of colloidal silica as the glidant; and
g) about 3.8 mg of magnesium stearate as the lubricant,
wherein the tablet weighs about 769 mg.

29. The fixed dose combination of claim 1, further comprising a film coat, the film coat comprising:
(a) a film former selected from the group consisting of hydroxypropyl methylcellulose (Hypromellose; HPMC), hydroxyprop yl cellulose (HPC), polyvinyl alcohol, and polyethylene glycol-polyvinyl alcohol (PEG-PVA) graft copolymer;
(b) a component selected from the group consisting of lactose monohydrate, mannitol, polydextrose, and isomalt; and
(c) a plasticizer selected from the group consisting of triacetin, polyethylene glycol, and a medium chain triglyceride; and
(d) an opacifier selected from the group consisting of calcium carbonate, rice starch, and titanium dioxide.

30. The fixed dose combination of claim 29, wherein the film coat comprises:
a first film coat comprising hypromellose, lactose monohydrate, triacetin, and either calcium carbonate or titanium dioxide at about 20-50%, about 10-25%, about 5-15%, and about 20-37.5% w/w, respectively, of the first film coat; and, optionally,
a second film coat underneath the first film coat, wherein the second film coat comprises hypromellose and triacetin in the ratio of about 88-93% to about 7.5-12%, respectively;
wherein the first and the second film coats lead to about 3-7% and about 0.5-3% w/w weight gain, respectively, of the fixed dose combination.

31-32. (canceled)

33. The fixed dose combination of claim 30, wherein the second film coat comprises hypromellose and triacetin at a ratio of about 91% to 9%.

34-39. (canceled)

40. A method of treating of HIV infection in a subject in need thereof, the method comprising administering the fixed dose combination of claim 1 to the subject.

41. (canceled)

42. The method of claim 40, wherein the fixed dose combination is administered once daily.

43. A method of treating of HIV infection in a subject in need thereof, the method comprising administering the tablet of claim 28 to the subject.

44-61. (canceled)

62. A pharmaceutical composition comprising a fixed dose combination for oral administration comprising 100 mg of doravirine in an amorphous solid dispersion of doravirine and a polymer and 0.25 mg of islatravir monohydrate.

63-66. (canceled)
